(19) United States
(12) Patent Application Publication
(10) Pub. No.: US 2011/0020461 A1
(43) Pub. Date: Jan. 27, 2011

(54) HYALURONATE AND COLOSTRUM COMPOSITIONS AND METHODS OF USING THE SAME

(76) Inventor: Harry Leneau, Jasper, MO (US)

Correspondence Address: ICE MILLER LLP, ONE AMERICAN SQUARE, SUITE 3100, INDIANAPOLIS, IN, 46282-0200, US

(21) Appl. No.: 12/509,876

(22) Filed: Jul. 27, 2009

(51) Int. Cl. A61K 35/20

(52) U.S. Cl. 424/535

(57) ABSTRACT

The present disclosure includes disclosure of various embodiments of composition comprising a therapeutically-effective amount of a hyaluronate and a therapeutically-effective amount of colostrum. In at least one embodiment, the hyaluronate comprises sodium hyaluronate, and the colostrum comprises bovine colostrum. In an exemplary method of reducing osteoblastic activity within a warm-blooded vertebrate, the method comprises the step of administering a composition comprising a therapeutically-effective amount of a hyaluronate and a therapeutically-effective amount of colostrum to the warm-blooded vertebrate.

### BACKGROUND

Arthritic disorders, including acute rheumatoid arthritis, chronic rheumatoid arthritis, and osteoarthritis, as well as various inflammatory skeletal and musculoskeletal conditions, affect millions of people, it has been previously estimated that 80% of all individuals over the age of 55 suffer from some form of arthritic disorder. Various animals, including equities, canines, and felines, also suffer from various arthritic disorders.

One of the most common arthritic disorders is osteoarthritis, developing gradually over time in many cases. Patients experience alternating periods of mild to moderate pain, stiffness, and swelling of the joint and periods of relatively symptom-free joint activity. Osteoarthritis is typically characterized by the deterioration of cartilage that covers the ends of bones at a joint, such as the knee or hip. In the healthy joint, cartilage acts as a shock absorber and aids the joint in bearing the stress of physical movement. In addition, synovial joint fluid produced by the synovial membrane lubricates the joint providing a slippery surface over which the bones may move. But as cartilage deteriorates, the bones begin to rub against each other causing joint pain. At the same time, the concentration of hyaluronic acid in the synovial joint decreases, reducing the lubrication ability of the synovial joint fluid. In addition, joint movement may be restricted as bone ends erode or thicken, and the bones may develop painful outgrowths, or bone spurs, as a result of this erosion or thickening. If left untreated, cartilage deterioration can seriously weaken the joint, possibly to the point of deformity.

Fibromyalgia, is a common disabling disorder characterized by chronic musculoskeletal aches and pain, stiffness, general fatigue, and sleep abnormalities. Fibromyalgia affects 2-4% of the population and is most frequently found in women between 20 and 50 years old. The exact cause of fibromyalgia remains uncertain, and diagnosis is difficult due to the general nature of the symptoms. Currently, the most effective treatment for fibromyalgia includes a combination of analgesics, sleep aids, exercise programs, relaxation techniques and other measures to reduce muscle tension. These treatments are geared toward improving sleep quality and reducing pain.

Current methods of reducing pain in osteoarthritic joints include treatment with analgesics or anti-inflammatory medications, physical therapy, topical application of hyaluronic acid to the joint, and intra-articular injection of hyaluronic acid directly into the joint. The primary goal of treatment is reduction of pain and maintenance of joint function and strength. Intra-articular injections of hyaluronic acid, known as viscosupplementation, have seen wide use for patients who have not responded well to other therapies. In addition, oral administration of hyaluronic acid has been disclosed as treatments for osteoarthritis.

As the bones of younger mammals grow, or as the bones of older mammals are being repaired, high levels of osteoblastic activity may be determined by, for example, testing for total alkaline phosphatase and/or the percentage of bone isoenzyme present within the total alkaline phosphatase test result. A decrease in osteoblastic activity in older horses, for example, would be considered indicative of healthy, or relatively healthier, bones.

Colostrum is a substance secreted in the first few days post-partum prior to onset of true lactation. Colostrum contains a number of proteins, carbohydrates, fats, vitamins, and minerals. In addition, colostrum contains bioactive components such as growth factors and antimicrobial factors. The antimicrobial factors include immunoglobulins, lactoperoxidase, lysozyme, and lactoferrin. Bovine colostrum is extremely rich in immunoglobulins. The concentration of IgG1 (52-87 g/l), IgG2 (1.6-2.1 g/l), IBM (3.7-6.1 g/l), and Riga (3.2-6.2 g/l) in bovine colostrum is approximately 100 fold higher than in normal bovine milk. However, colostrum, by its nature, is not a sterile product, and its use has been generally limited to oral ingestion.

The disclosure of the present application provides various compositions comprising a hyaluronate and a colostrum useful for treating, for example, osteoarthritis, showing beneficial synergistic results previously unknown to the public. Such beneficial results may be indicated by, for example, a decrease in alkaline phosphatase and/or the percentage of bone isoenzyme present within the total alkaline phosphatase test result over time.

### BRIEF SUMMARY

In at least one embodiment of composition of the present disclosure, the composition comprises therapeutically-effective amounts of a hyaluronate and colostrum. In various embodiments, the hyaluronate consists of hyaluronic acid, sodium hyaluronate, another hyaluronate salt, or a hyaluronate digest. In at least one embodiment, the colostrum comprises bovine colostrum. In various embodiments, exemplary compositions, or portions thereof, may be sterilized and/or highly filtered.

In at least one embodiment of composition of the present disclosure, the composition further comprises a food acceptable carrier. Exemplary food acceptable carriers may include any number of foods and/or water. In at least one embodiment, the food acceptable carrier comprises water and/or molasses.

Compositions of the present disclosure may be provided in various forms, including capsule/gel seal form, liquid form, or paste form. Additional embodiments may be provided in injectable form for subsequent injection.

In at least one embodiment of a nutritional supplement of the present disclosure, die nutritional supplement comprises a therapeutically-effective amount of a hyaluronate, or a salt or digest thereof, a therapeutically-effective amount of colostrum, and a food acceptable carrier, wherein the nutritional supplement is provided in an orally ingestible dosage form. In at least one embodiment of such a nutritional supplement, the orally ingestible dosage form comprises a capsule or gel seal. In another embodiment, the orally ingestible dosage form consists of a liquid form, a paste form, or an injectable form.

In at least one method of reducing osteoblastic activity within a warm-blooded vertebrate of the present disclosure, the method comprises the step of administering a composition comprising a therapeutically-effective amount of a hyaluronate and a therapeutically-effective amount of colostrum. In an exemplary embodiment, the warm-blooded vertebrate is chosen from a human, an equine species, a canine species, or a feline species. In at least one embodiment, the colostrum is obtained from an animal of the same or other species as the warm-blooded vertebrate. In various embodiments, the step of administering comprises oral administration or subcutaneous injection.

### DETAILED DESCRIPTION

The disclosure of the present application comprises several embodiments of a composition comprising a hyaluronate and a colostrum, wherein said composition is useful to treat warm-blooded vertebrates. In an exemplary embodiment, sodium hyaluronate, the sodium salt of hyaluronic acid, is used as the hyaluronate, and bovine colostrum is used as the colostrum. In various other embodiments, any number of hyaluronates, such as other hylauronate salts, digests, or hyaluronic acid itself, may be used as the hyaluronate, and any number of mammalian colostrums may be used as the colostrum. Such exemplary compositions of the disclosure of the present application may be useful for treating joint pain or other discomforts associated with joint disorders, including osteoarthritis, rheumatoid arthritis, and fibromyalgia.

Exemplary compositions of the disclosure of the present application may be prepared by combining an amount of a hyaluronate, an amount of colostrum, and a food acceptable carrier. Exemplary food acceptable carriers may comprise any number of foods, including molasses, traditional animal/livestock feed, and/or a liquid such as water. Such compositions may be prepared in any number of forms, such as capsule/gel seal form, liquid form, paste form, and/or as a mixture with one or more food acceptable carriers.

Compositions of the disclosure of the present application may be useful to treat any number of arthritic disorders, including, but not limited to, acute rheumatoid arthritis, chronic rheumatoid arthritis, osteoarthritis, as well as various inflammatory skeletal and musculoskeletal conditions, including fibromyalgia.

Furthermore, and as demonstrated in the examples herein, the synergistic effect of the combination of a hyaluronate and colostrum shows improved treatment for one or more of the aforementioned conditions above and beyond the use of a hyaluronate or a colostrum by itself. For example, an exemplary composition of the present application may be used to treat equines for joint pain and/or discomfort in lieu of a treatment comprising a hyaluronate without colostrum, providing an improved treatment outcome above and beyond therapeutics currently known and used in the art.

### EXAMPLE 1

### Safety Testing

In an exemplary canine and equine test for safety, a mixture of 20% bovine colostrum and hyaluronic acid was prepared and administered to canines and equines.

To determine the effects of bovine colostrum and hyaluronic acid on canines, twenty-three (23) canines were administered 4 mL of a product supplying 10 mg/mL sodium hyaluronate and containing 20% colostrum per day for a period of six (6) weeks. The 4 mL dosage was a cumulative dosage of 2 mL in the morning and 2 mL in the evening. No adverse reactions were seen or reported in any of the 23 canines tested.

In addition, and to determine the effects of bovine colostrum and hyaluronic acid on horses, thirteen (13) horses were administered 4 mL of the same product per day for a period of six (6) weeks. The 4 mL dosage was also a cumulative dosage of 2 mL in the morning and 2 mL in the evening. No adverse reactions were seen or reported in any of the 13 horses.

### EXAMPLE 2

### Exemplary Product Testing

Four batches of an exemplary formulation of the disclosure of the present application were prepared for potential testing in warm-blooded vertebrates:

Batch 1: 22 grams of sodium hyaluronate was mixed in 1800 mL of water. 400 mL of bovine colostrum was added to the sodium hyaluronate mixture. The final concentrations of the combined 2200 mL mixture were 10.00 mg/ml sodium hyaluronate and 18.18% bovine colostrum.

Batch 2: 20 grams of sodium hyaluronate was mixed in 1800 mL of water. 200 mL of bovine colostrum was added to the sodium hyaluronate mixture. The final concentrations of the combined 2000 mL mixture were 10.00 mg/mL sodium hyaluronate and 10.00% bovine colostrum.

Batch 3: 19 grams of sodium hyaluronate was mixed in 1800 mL of water. 100 mL of bovine colostrum was added to the sodium hyaluronate mixture. The final concentrations of the combined 1900 mL mixture were 10.00 mg/mL sodium hyaluronate and 5.26% bovine colostrum.

Batch 4: 72 grams of sodium hyaluronate was mixed in 1800 mL of water. 400 mL of bovine colostrum was added to the sodium hyaluronate mixture. The final concentrations of the combined 2200 mL mixture were 32.72 mg/mL sodium hyaluronate and 18.18% bovine colostrum.

An additional batch (“Batch 5”) was prepared as follows for use as a control for the testing referenced herein:

Batch 5: 20 grams of sodium hyaluronate was mixed in 2000 mL of water. The final concentration of the mixture was 10.00 mg/mL sodium hyaluronate.

### Alkaline Phosphatase Total+Isoenzymes

Twenty-three (23) horses were tested using either the aforementioned Batch 2 product or a control product (referred to herein as Batch 5, Amvets serial no. 1001, comprising 9 mg/mL sodium hyaluronate). Regarding the 23 horses tested, thirteen (13) horses, numbered 1-13, were tested with a sodium hyaluronate and colostrum product. The remaining ten (10) horses, numbered 14-23, were tested with the Batch 5 product.

The characteristics of the 13 horses tested with the combined Batch 2 product are provided in Table 1 below:

TABLE 1

| Horse | | | |
|---|---|---|---|
| No. | Age | Breed | Gender/Type |
| 1 | 6 | Q | Mare |
| 2 | 9 | Q | Gelding |
| 3 | 14 | Q | Gelding |
| 4 | 12 | Q | Gelding |
| 5 | 7 | Q | Stallion |
| 6 | 31 | T | Gelding |
| 7 | 6 | T | Gelding |
| 8 | 19 | M | Gelding |
| 9 | 17 | M | Gelding |
| 10 | 14 | TMix | Mare |
| 11 | 17 | TMix | Gelding |
| 12 | 15 | P | Gelding |
| 13 | 15 | M | Gelding |

The characteristics of the 10 horses tested with the Batch 5 control product are provided in Table 2 below:

TABLE 2

| Horse | | | |
|---|---|---|---|
| No. | Age | Breed | Gender/Type |
| 14 | 7 | M | Mare |
| 15 | 10 | Mix | Mare |
| 16 | 6 | Q | Gelding |
| 17 | 8 | Q | Gelding |
| 18 | 7 | Q | Gelding |
| 19 | 9 | Q | Stallion |
| 20 | 8 | Q | Mare |
| 21 | 8 | Q | Gelding |
| 22 | 13 | Qmix | Gelding |
| 23 | 10 | Q | Gelding |

As referenced within Tables 1 and 2 above, the age of the horses is given in years, and the various horse breeds listed are as follows:
M=Morgan
Mix=No specific breed
P=Paint
Q=American Quarter Horse
Qmix=America Quarter Horse mix
T=Thoroughbred
Tmix=Thoroughbred mix

To obtain baseline data, total alkaline phosphatase baselines were determined for each of the 23 tested horses. In this particular example, and to determine total alkaline phosphatase (in International Units), a Heska analyzer was used to obtain the total alkaline phosphatase (Dri Chem 4000), and a Helena electrophoresis system was used to separate the isoenzyme bands of the alkaline phosphatase. In at least one other embodiment, various other methods and/or equipment may be used to determine total alkaline phosphatase (in International Units) from obtained samples.

Baseline data obtained for the 23 horses is shown in Table 3 below:

TABLE 3

| Horse | Total Alk. |
|---|---|
| No. | Phos. (I.U.) |
| 1 | 291 |
| 2 | 198 |
| 3 | 207 |
| 4 | 222 |
| 5 | 314 |
| 6 | 174 |
| 7 | 203 |
| 8 | 195 |
| 9 | 177 |
| 10 | 159 |
| 11 | 205 |
| 12 | 214 |
| 13 | 180 |
| 14 | 153 |
| 15 | 262 |
| 16 | 260 |
| 17 | 188 |
| 18 | 185 |
| 19 | 197 |
| 20 | 248 |
| 21 | 223 |
| 22 | 197 |
| 23 | 289 |

In addition to determining the total alkaline phosphatase (as referenced in Table 3 above), the percentage of isoenzyme pertaining specifically to bone was also determined. The bone fraction, separated from the liver fraction and potentially other smaller tractions, would be considered as an indicator pertaining to osteoblastic activity. For example, if a high bone fraction were present, it would be indicative of high osteoblastic activity. Similarly, if the bone fraction of total alkaline phosphatase were to decrease over time in response to a treatment, it would be indicative of reduced osteoblastic activity. Applicants hypothesized that if reduced osteoblastic activity was seen over time in response to a particular treatment, it would be indicative of the positive effects of the particular treatment, as the mammal being treated would not have a need for as-frequent bone growth. Specifically, reduced osteoblastic activity in older horses in response to a treatment would be indicative of generally stronger and more durable bones within the horse.

In this particular example, the baseline bone fractions were determined via separation of the various isoenzyme bands by electrophoresis. The baseline bone fractions for the 23 horses in this example are as shown in Table 4 below:

TABLE 4

| Horse | Baseline % |
|---|---|
| No. | Bone (I.U.) |
| 1 | 63 |
| 2 | 57 |
| 3 | 66 |
| 4 | 69 |
| 5 | 77 |
| 6 | 59 |
| 7 | 55 |
| 8 | 61 |
| 9 | 52 |
| 10 | 47 |
| 11 | 66 |
| 12 | 61 |
| 13 | 47 |
| 14 | 55 |
| 15 | 61 |
| 16 | 67 |
| 17 | 52 |
| 18 | 53 |
| 19 | 60 |
| 20 | 66 |
| 21 | 58 |
| 22 | 61 |
| 23 | 78 |

The 23 horses were then treated as follows: horses 1-13 were treated with the Batch 2 product referenced above, and horses 14-23 were treated with the Batch 5 control product referenced above. The horses were provided 2 mL of their respective formulations twice a day for four (4) weeks.

After two (2) weeks, total alkaline phosphatase tests were performed to determine the effect, if any, of the two formulations on the tested horses. In addition, the percentage of isoenzyme pertaining to the bone was also determined. Week two total alkaline phosphatase data for the 23 horses is shown in Table 5 below:

TABLE 5

| Horse | Total Alk. |
|---|---|
| No. | Phos. (I.U.) |
| 1 | 181 |
| 2 | 113 |
| 3 | 138 |
| 4 | 154 |
| 5 | 243 |
| 6 | 102 |
| 7 | 111 |
| 8 | 119 |
| 9 | 92 |
| 10 | 77 |
| 11 | 135 |
| 12 | 130 |
| 13 | 85 |
| 14 | 84 |
| 15 | 160 |
| 16 | 174 |
| 17 | 98 |
| 18 | 87 |
| 19 | 101 |
| 20 | 150 |
| 21 | 110 |
| 22 | 112 |
| 23 | 178 |

Week two data pertaining to the percentage of bone isoenzyme is shown in Table 6 below:

TABLE 6

| Horse | % Bone ISO |
|---|---|
| No. | (I.U.) |
| 1 | 51 |
| 2 | 41 |
| 3 | 50 |
| 4 | 52 |
| 5 | 62 |
| 6 | 42 |
| 7 | 43 |
| 8 | 57 |
| 9 | 40 |
| 10 | 36 |
| 11 | 54 |
| 12 | 49 |
| 13 | 33 |
| 14 | 43 |
| 15 | 49 |
| 16 | 61 |
| 17 | 47 |
| 18 | 44 |
| 19 | 51 |
| 20 | 60 |
| 21 | 47 |
| 22 | 57 |
| 23 | 62 |

Similarly, and after four weeks of testing, total alkaline phosphatase tests and percentage of isoenzyme pertaining to the bone tests were performed to determine the effect of the two formulations. Week four total alkaline phosphatase data is shown in Table 7 below:

TABLE 7

| Horse | Total Alk. |
|---|---|
| No. | Phos. (I.U.) |
| 1 | 95 |
| 2 | 84 |
| 3 | 79 |
| 4 | 73 |
| 5 | 124 |
| 6 | 69 |
| 7 | 78 |
| 8 | 67 |
| 9 | 74 |
| 10 | 68 |
| 11 | 81 |
| 12 | 67 |
| 13 | 51 |
| 14 | 49 |
| 15 | 86 |
| 16 | 101 |
| 17 | 61 |
| 18 | 57 |
| 19 | 56 |
| 20 | 68 |
| 21 | 57 |
| 22 | 60 |
| 23 | 112 |

Table 8 below shows the week four data pertaining to the percentage of bone isoenzyme:

TABLE 8

| Horse | % Bone ISO |
|---|---|
| No. | (I.U.) |
| 1 | 44 |
| 2 | 38 |
| 3 | 48 |
| 4 | 47 |
| 5 | 51 |
| 6 | 37 |
| 7 | 38 |
| 8 | 46 |
| 9 | 31 |
| 10 | 31 |
| 11 | 45 |
| 12 | 50 |
| 13 | 34 |
| 14 | 41 |
| 15 | 49 |
| 16 | 58 |
| 17 | 44 |
| 18 | 41 |
| 19 | 50 |
| 20 | 62 |
| 21 | 46 |
| 22 | 51 |
| 23 | 64 |

The raw data shown in Tables 3-8 herein was then used to prepare the following table (Table 9) showing the raw decrease in percentage of bone isoenzyme from the baseline to the four-week period:

TABLE 9

| Horse | 4 Week Decrease | Horse | 4 Week Decrease |
|---|---|---|---|
| No. | in % bone ISO | No. | in % bone ISO |
| 1 | 19 | 14 | 14 |
| 2 | 19 | 15 | 12 |
| 3 | 18 | 16 | 9 |
| 4 | 22 | 17 | 8 |
| 5 | 26 | 18 | 12 |
| 6 | 22 | 19 | 10 |
| 7 | 17 | 20 | 4 |
| 8 | 15 | 21 | 12 |
| 9 | 21 | 22 | 10 |
| 10 | 16 | 23 | 14 |
| 11 | 21 | | |
| 12 | 11 | | |
| 13 | 13 | | |
| Avg. Dec. | 18.5 | Avg. Dec. | 10.5 |
| Std. Dev. | 4.1 | Std. Dev. | 3 |

As shown in Table 9, the average decrease in the percentage of bone isoenzyme over four weeks for the horses treated with the sodium hyaluronate and colostrum combination was 18.5, with a standard deviation of 4.1. Conversely, the average decrease in the percentage of bone isoenzyme over the same time period for the horses treated with only sodium hyaluronate was 10.5, with a standard deviation of 3. The differences between the two decreases is approximately two standard deviations away from the larger figure, demonstrating a significant statistical difference between the horses treated with an exemplary composition of the present disclosure and horses treated only with a hyaluronic acid composition.

While various embodiments of compositions comprising a hyaluronate and colostrum and methods of using the same have been described in considerable detail herein, the embodiments are merely offered by way of non-limiting examples of the disclosure described herein. Many variations and modifications of the embodiments described herein will be apparent to one of ordinary skill in the art in light of this disclosure. It will therefore be understood by those skilled in the art that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations of the present disclosure. In addition, disclosure directed to a method and/or process should not be limited to the performance of their steps in the order written, and one skilled in the art can readily appreciate that the sequences may be varied and still remain within the spirit and scope of the present disclosure.

It is therefore intended that the disclosure will include all modifications and changes apparent to those of ordinary skill in the art based on this disclosure.

1. A composition, comprising:
a therapeutically-effective amount of a hyaluronate; and
a therapeutically-effective amount of a colostrum.

2. The composition of claim 1, wherein the hyaluronate comprises at least one form of hyaluronate chosen from hyaluronic acid, sodium hyaluronate, a hyaluronate salt, or a hyaluronate digest.

3. The composition of claim 1, wherein the colostrum comprises bovine colostrum.

4. The composition of claim 1, wherein at least a portion of the composition is highly filtered.

5. The composition of claim 1, further comprising a food acceptable carrier.

6. The composition of claim 5, wherein the food acceptable carrier is at least one carrier chosen from food, water, or molasses.

7. The composition of claim 1, wherein the composition is provided in a form chosen from a capsule form, a gel seal form, a liquid form, a paste form, or an injectable form.

8. A nutritional supplement, comprising;
a therapeutically-effective amount of a hyaluronate, or an acid, a salt, or a digest thereof;
a therapeutically-effective amount of a colostrum; and
a food acceptable carrier;
wherein the nutritional supplement is provided in an orally ingestible dosage form.

9. The nutritional supplement of claim 8, wherein the orally ingestible dosage form is chosen from a capsule form, a gel seal form, a liquid form, or a paste form.

10. A method of treating a warm-blooded vertebrate, the method comprising the step of administering a composition comprising a therapeutically-effective amount of a hyaluronate and a therapeutically-effective amount of colostrum to the warm-blooded vertebrate.

11. The method of claim 10, wherein the step of administering the composition is performed to deliver a composition capable of reducing osteoblastic activity within the warm-blooded vertebrate to the warm-blooded vertebrate.

12. The method of claim 10, wherein the step of administering the composition is performed to deliver the composition which is capable of treating a disorder, the disorder chosen from acute rheumatoid arthritis, chronic rheumatoid arthritis, osteoarthritis, or fibromyalgia.

13. The method of claim 10, wherein the hyaluronate comprises a hyaluronate chosen from hyaluronic acid, sodium hyaluronate, a hyaluronate salt, or a hyaluronate digest.

14. The method of claim 10, wherein the warm-blooded vertebrate is chosen from a human, an equine species, a canine species, or a feline species,

15. The method of claim 10, wherein the colostrum is obtained from an animal of the same species as the warm-blooded vertebrate.

16. The method of claim 10, wherein the colostrum is obtained from an animal of a different species as the warm-blooded vertebrate,

17. The method of claim 10, wherein the step of administering comprises oral administration.

18. The method of claim 10, wherein at least a portion of the composition is highly filtered.

19. The method of claim 10, wherein the composition further comprises a food acceptable carrier, the food acceptable carrier chosen from food, water, molasses, or traditional animal/livestock feed.

20. The method of claim 10, wherein the composition is provided in a form chosen from a capsule form, a gel seal form, a liquid form, a paste form, and an injectable form.
